(19) United States
(12) Patent Application Publication
(10) Pub. No.: US 2025/0135233 A1
(43) Pub. Date: May 1, 2025

(54) Target Volume Monitoring Method, Electronic Apparatus and Radiotherapy System

(71) Applicant: OUR UNITED CORPORATION, Shaanxi (CN)

(72) Inventors: Hao Yan, Shaanxi (CN); Haifeng Liu, Shaanxi (CN); Zhaohui Zhu, Shaanxi (CN)

(21) Appl. No.: 18/930,810

(22) Filed: Oct. 29, 2024

(30) Foreign Application Priority Data
Oct. 30, 2023 (CN) 202311427079.1

(51) Int. Cl. A61N 5/10; G06T 7/30; G06T 7/70

(52) U.S. Cl. CPC A61N 5/1049; G06T 7/30; G06T 7/70; G06T 2200/04; G06T 2207/10081; G06T 2207/30096

(57) ABSTRACT

A target volume monitoring method includes obtaining at least one two-dimensional slice image of a target object from an imaging apparatus, where any one of the at least one two-dimensional slice image includes a desired target volume of the target object; obtaining a three-dimensional reference image of the target object; and registering the at least one two-dimensional slice image with the three-dimensional reference image to obtain a registration result, so as to monitor a position of the desired target volume.

### CROSS-REFERENCE TO RELATED APPLICATION

This application claims priority to Chinese Patent Application No. 202311427079.1, filed Oct. 30, 2023, the disclosure of which is hereby incorporated by reference in its entirety.

### BACKGROUND OF THE INVENTION

### Field of the Invention

The present disclosure relates to the technical field of radiotherapy, and in particular, to a target volume monitoring method, an electronic apparatus and a radiotherapy system.

### Description of Related Art

With the rapid development of computer and imaging technology, radiotherapy technology has continued to mature. Image-guided radiation therapy (IGRT) is used to monitor a target volume of a patient, so that an accuracy of radiotherapy may be effectively ensured, a local control rate of tumors may be improved, and a radiation dose to surrounding normal tissues may be reduced.

Before and during radiotherapy, cone-beam computed tomography (CBCT) imaging is generally used to monitor the position of the target volume of the patient. Here, CBCT imaging may generate a two-dimensional projection image containing the target volume of the patient, and may also reconstruct a three-dimensional reconstructed image containing the target volume of patient through two-dimensional projection images collected in a plurality of directions.

### SUMMARY OF THE INVENTION

In an aspect, a target volume monitoring method is provided. The method includes: obtaining at least one two-dimensional slice image of a target object from an imaging apparatus, where any one of the at least one two-dimensional slice image includes a desired target volume of the target object; obtaining a three-dimensional reference image of the target object; and registering the at least one two-dimensional slice image with the three-dimensional reference image to obtain a registration result, so as to monitor a position of the desired target volume.

In another aspect, a target volume monitoring electronic apparatus is provided. The target volume monitoring electronic apparatus includes: at least one processor, and a memory communicatively connected to the at least one processor. The memory stores instructions executable by the at least one processor, and the instructions is executed by the at least one processor, so that the at least one processor is capable of executing: obtaining at least one two-dimensional slice image of a target object from an imaging apparatus, where any one of the at least one two-dimensional slice image includes a desired target volume of the target object; obtaining a three-dimensional reference image of the target object; and registering the at least one two-dimensional slice image with the three-dimensional reference image to obtain a registration result, so as to monitor a position of the desired target volume.

In yet another aspect, a non-transitory computer-readable storage medium storing computer program instructions is provided. The computer program instructions, when executed by an electronic apparatus, cause the electronic apparatus to implement the target volume monitoring method according to some embodiments of the present disclosure.

In yet another aspect, a computer program product is provided. The computer program product includes a computer program that, when executed by a processor, cause the processor to implement the target volume monitoring method according to some embodiments of the present disclosure.

In yet another aspect, a radiotherapy system is provided. The radiotherapy system includes a radiotherapy apparatus, an imaging apparatus, a target volume monitoring electronic apparatus, and a radiotherapy control electronic apparatus. The imaging apparatus is configured to generate at least one two-dimensional slice image of a target object. The target volume monitoring electronic apparatus is configured to execute the above target volume monitoring method and output a registration result. The radiotherapy control electronic apparatus is configured to obtain the registration result and control operation of the radiotherapy apparatus according to the registration result.

It should be understood that what is described in the summary is not intended to identify critical or significant features of the embodiments of the disclosure, nor is it intended to limit the scope of the present disclosure. Other features of the present disclosure will become readily understood from the following description.

### BRIEF DESCRIPTION OF THE DRAWINGS

The accompanying drawings are used to well understand the present solution and do not constitute a limitation of the present disclosure.

FIG. 1A is a schematic diagram of an implementation environment of a target volume monitoring method, in accordance with some embodiments of the present disclosure;

FIG. 1B is a schematic diagram of another implementation environment of the target volume monitoring method, in accordance with some embodiments of the present disclosure;

FIG. 1C is a schematic diagram of yet another implementation environment of the target volume monitoring method, in accordance with some embodiments of the present disclosure;

FIG. 2 is a schematic flow chart of a target volume monitoring method, in accordance with some embodiments of the present disclosure;

FIG. 3 is a schematic flow chart of a target volume monitoring method used in a setup phase, in accordance with some embodiments of the present disclosure;

FIG. 4 is a schematic diagram of a flow chart of a target volume monitoring method used in a radiotherapy phase, in accordance with some embodiments of the present disclosure; and

FIG. 5 is a block diagram of a target volume monitoring electronic apparatus used to implement the target volume monitoring method, in accordance with some embodiments of the present disclosure.

### DESCRIPTION OF THE INVENTION

Some embodiments of the present disclosure will be clearly and completely described below with reference to the accompanying drawings. However, the described embodiments are merely some but not all embodiments of the present disclosure. All other embodiments obtained by a person of ordinary skill in the art based on embodiments of the present disclosure shall be included in the protection scope of the present disclosure.

Unless the context requires otherwise, throughout the specification and the claims, the term “comprise” and other forms thereof such as the third-person singular form “comprises” and the present participle form “comprising” are construed as an open and inclusive meaning, i.e., “including, but not limited to.” In the description of the specification, the terms such as “some embodiments”, “example” or “some examples” are intended to indicate that specific features, structures, materials or characteristics related to the embodiment(s) or example(s) are included in at least one embodiment or example of the present disclosure. Schematic representation of the above terms do not necessarily refer to the same embodiment(s) or example(s). In addition, the specific features, structures, materials or characteristics may be included in any one or more embodiments or examples in any suitable manner.

Hereinafter, the terms “first” and “second” are used for descriptive purposes only, and are not to be construed as indicating or implying the relative importance or implicitly indicating the number of indicated technical features. Thus, features defined with “first” or “second” may explicitly or implicitly include one or more of the features. In the description of the embodiments of the present disclosure, the term “a plurality of/the plurality of” means two or more unless otherwise specified.

In the description of some embodiments, the terms such as “connected” and derivatives thereof may be used. The term “connected” should be understood in a broad sense. For example, the term “connected” may represent a fixed connection, a detachable connection, or a one-piece connection, or may represent a direct connection, or may represent an indirect connection through an intermediate medium. The embodiments disclosed herein are not necessarily limited to the contents herein.

The phrase “at least one of A, B and C” has a same meaning as the phrase “at least one of A, B or C”, and they both include the following combinations of A, B and C: only A, only B, only C, a combination of A and B, a combination of A and C, a combination of B and C, and a combination of A, B and C.

As used herein, the term “if” is optionally construed as “when” or “in a case where” or “in response to determining” or “in response to detecting”, depending on the context. Similarly, depending on the context, the phrase “if it is determined . . . ” or “if [a stated condition or event] is detected” is optionally construed as “in a case where it is determined . . . ”, “in response to determining . . . ”, “in a case where [the stated condition or event] is detected” or “in response to detecting [the stated condition or event]”.

The use of the phrase “configured to” herein means an open and inclusive language, which does not exclude devices that are applicable to or configured to perform additional tasks or steps.

In addition, the use of the phrase “based on” is meant to be open and inclusive, since a process, step, calculation or other action that is “based on” one or more of the stated conditions or values may, in practice, be based on additional conditions or values beyond those stated.

The term such as “parallel,” or “perpendicular,” as used herein includes a stated condition and a condition similar to the stated condition. A range of the similar condition is within an acceptable deviation range, and the acceptable deviation range is determined by a person of ordinary skill in the art, considering measurement in question and errors associated with measurement of a particular quantity (i.e., the limitations of a measurement system). For example, the term “parallel” includes absolute parallelism and approximate parallelism, and an acceptable range of deviation of the approximate parallelism may be, for example, a deviation within 5°; the term “perpendicular” includes absolute perpendicularity and approximate perpendicularity, and an acceptable range of deviation of the approximate perpendicularity may also be, for example, a deviation within 5°.

Exemplary embodiments of the present disclosure are described below with reference to the drawings, in which various details of the embodiments of the present disclosure are included to facilitate understanding, and these embodiments should be considered as exemplary only. Therefore, those skilled in the art should realize that various changes and modifications can be made to the embodiments described herein without departing from the scope of the present disclosure. Similarly, for clarity and conciseness, description of well-known functions and structures have been omitted from the following description.

Before and during radiotherapy, CBCT is usually used to generate a two-dimensional projection image containing the target volume of the patient. When necessary, it may be possible to reconstruct a three-dimensional reconstructed image containing the target volume of the patient from two-dimensional projection images collected in a plurality of directions.

However, although the two-dimensional projection image has a fast imaging speed, it has a problem of low image quality, which in turn affects the accuracy of target volume monitoring. In addition, although the three-dimensional reconstructed image of CBCT may directly reflect a three-dimensional volume structure of the target volume of the patient and its surroundings, the image acquisition and reconstruction process may take a long time and cannot be used for real time monitoring of the target volume.

First, the application scenarios involved in some embodiments of the present disclosure are described. The target volume monitoring method provided by some embodiments of the present disclosure may be applied in the field of medical technology, for example, in scenarios with image-guided radiotherapy. In some embodiments, the target volume monitoring method provided by some embodiments of the present disclosure may be applied to at least one of a setup phase before radiotherapy or a radiotherapy phase during radiotherapy.

During the setup phase before radiotherapy, a target object (e.g., the tumor target volume of the patient) is positioned at a target position, which may be an isocenter of the radiotherapy apparatus (or a virtual isocenter having a preset positional relationship with the isocenter of the radiotherapy apparatus). During the radiotherapy phase of radiotherapy, the target object is monitored in real time to ensure that the tumor target volume is within the preset range of the target position.

In general, real time monitoring during the positioning or radiotherapy of the target object may be achieved by using CBCT imaging.

During the setup phase before radiotherapy, positioning the target volume of the target object at the virtual isocenter may be implemented in the following two methods.

In a method of positioning, step (A1) to step (A3) need to be performed.

In step (A1), the CBCT apparatus obtains two two-dimensional projection images including the tumor target volume of the target object in two preset directions. For example, the two preset directions are directions with a gantry angle of 0 degree and 90 degree, respectively.

In step (A2), the target volume monitoring electronic apparatus (e.g., a server) obtains the two-dimensional projection images and performs image registration on the two-dimensional projection images with reference images in corresponding directions, so as to obtain an offset indicating a magnitude of the position offset of the target volume.

Here, the reference images may be two digitally reconstructed radiograph (DRR) images in two preset directions, and the two digitally reconstructed radiograph images are generated according to a treatment planning (e.g., a computed tomography (CT) image) of the target object. The offset is an offset in at least three coordinate axis directions in an International Electrotechnical Commission (IEC) coordinate system.

In step (A3), in a case where a radiotherapy control electronic apparatus obtains the offset, and if it is determined that the offset is outside a preset threshold range, the radiotherapy apparatus is instructed to adjust a position of a treatment couch, and step (A1) is re-performed until the offset is within the preset threshold range. In this way, the positioning of the target object may be completed based on the two-dimensional projection image including the target volume of the target object.

In another implementation of positioning, the following step (B1) to step (B3) need to be performed.

In step (B1), the CBCT apparatus obtains a plurality of two-dimensional projection images including a tumor target volume of the target object in a plurality of preset different directions. For example, any one of the plurality of preset different directions is a direction corresponding to any angle in a range from 0 degrees to 280 degrees of the gantry angle.

In step (B2), the target volume monitoring electronic apparatus (e.g., a server) obtains the plurality of two-dimensional projection images, performs three-dimensional reconstruction on the plurality of two-dimensional projection images to obtain a three-dimensional reconstructed image including the target volume of the target object, and performs image registration on the three-dimensional reconstructed image and the reference image, so as to obtain an offset indicating the magnitude of the position offset of the target volume.

Here, the reference image may be generated according to a treatment planning image (e.g., a CT image) of the target object, and the offset is an offset in at least three coordinate axis directions (i.e., a horizontal direction) in the IEC coordinate system.

In step (B3), in a case where the radiotherapy control electronic apparatus obtains the offset, and determines that the offset is outside the preset threshold range, the radiotherapy apparatus is instructed to adjust the position of the treatment couch, and step (B1) is re-performed until the offset is within the preset threshold range. In this way, the positioning of the target object may be completed based on the three-dimensional reconstructed image containing the target volume of the target object.

During the radiotherapy phase of radiotherapy, monitoring of the target volume of the target object may be implemented through the following methods.

Assuming that, during radiotherapy, the CBCT apparatus collects a projection image once in a direction corresponding to each gantry angle interval of 1 degree. For example, projection images containing the target volume of the target object are collected in directions of gantry angles of 0 degree, 1 degree, 2 degree, 3 degree, etc. The projection images collected by the CBCT apparatus in the direction corresponding to each gantry angle need to be processed as follows, the processing includes step (C1) to step (C3).

In step (C1), the target volume monitoring electronic apparatus (e.g., a server) obtains a two-dimensional projection image in an i-th direction at a current moment, and performs the image registration on the two-dimensional projection image in the i-th direction with a corresponding reference image in the i-th direction, so as to obtain a two-dimensional offset corresponding to the i-th direction; the target volume monitoring electronic apparatus obtains a pre-stored two-dimensional projection image in a j-th direction, and performs image registration on the two-dimensional projection image in the j-th direction with the corresponding reference image in the j-th direction to obtain a two-dimensional offset corresponding to the j-th direction; and obtains a three-dimensional offset of the target volume of the target object at the current moment based on the two-dimensional offset corresponding to the i-th direction and the two-dimensional offset corresponding to the j-th direction.

Here, an included angle between the i-th direction and the j-th direction is 90 degrees. It can be understood that when the two-dimensional projection images are collected in directions corresponding to 1 degree to 89 degrees, it is impossible to monitor the target volume of the target object.

In step (C2), the radiotherapy control electronic apparatus obtains a three-dimensional offset of the target volume of the target object at the current moment, and controls the radiotherapy apparatus to stop emitting beams when it is determined that the three-dimensional offset of the target volume of the target object at the current moment is outside the preset threshold range. Otherwise, step (C1) is performed to continue to obtain the projection image collected in the direction corresponding to a next gantry angle and perform the above processing until the radiotherapy is completed.

In this way, the real time monitoring of the target object is completed based on the two-dimensional projection images of different directions corresponding to the target volume of the target object at different moments.

However, although the two-dimensional projection images of CBCT have fast imaging or acquisition speed, the image quality of the two-dimensional projection images is low, which affects the registration accuracy with the reference image and may affect the accuracy of the positioning monitoring or real time monitoring of the patient. In addition, although the three-dimensional reconstructed images of CBCT may directly reflect the three-dimensional volume structure of the target volume of the patient and its surroundings, the acquisition and reconstruction process takes a long time and may not be used for the real time monitoring of the target volume.

In view of this, some embodiments of the present disclosure provide a target volume monitoring method, which may quickly obtain a two-dimensional slice image including the desired target volume of the target object in at least one direction from an imaging apparatus (e.g., an MRI apparatus), and the two-dimensional slice image is used as a real time image and registered with the three-dimensional reference image of the target object, so as to monitor the position of the desired target volume. In this way, the accuracy of position monitoring of the target volume may be improved through high-quality two-dimensional slice images. In addition, the quickly obtained two-dimensional slice images may be used for positioning before radiotherapy and real time monitoring during radiotherapy, thereby improving the accuracy of radiotherapy.

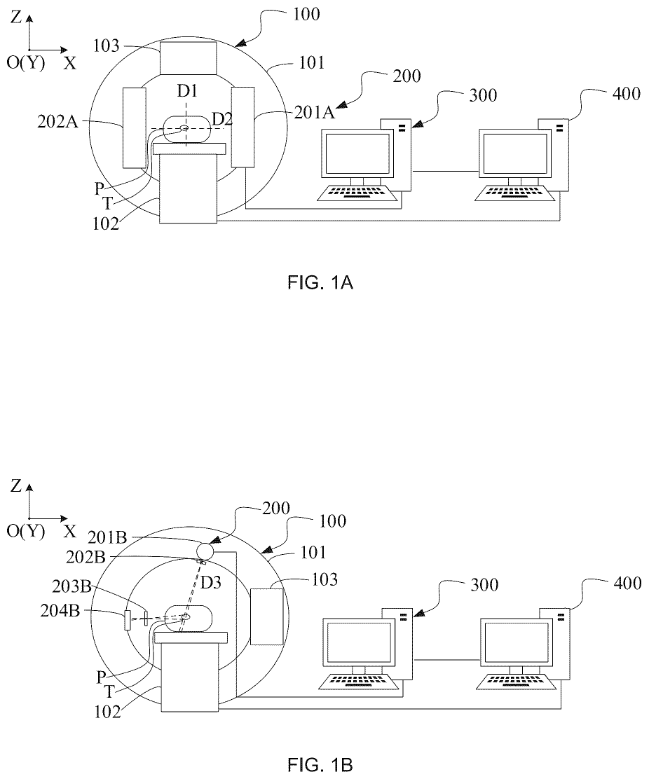

FIGS. 1A to 1C are schematic diagrams of different implementation environments of the target volume monitoring method, in accordance with some embodiments of the present disclosure. Referring to FIGS. 1A and 1B, the implementation environment includes a radiotherapy apparatus 100, an imaging apparatus 200, a target volume monitoring electronic apparatus 300, and a radiotherapy control electronic apparatus 400.

In some embodiments, the radiotherapy apparatus 100 may include a gantry 101, a positioning support device 102, and a radiotherapy head 103 disposed on the gantry 101.

For example, the gantry 101 may be a ring gantry, a C-arm gantry, a drum gantry, a multi-layer bowl-shaped or cylindrical structure gantry, or the like. The gantry 101 may drive the radiotherapy head 103 to rotate or not rotate. The positioning support device 102 may support and position the target object P, and the target object P may be a patient or a phantom simulating a patient.

For example, the positioning support device 102 may be a three-dimensional, four-dimensional, five-dimensional or six-dimensional treatment couch or treatment chair, etc. The radiotherapy head 103 may emit a beam to perform radiotherapy on the tumor target volume of the target object. The beam emitted by the radiotherapy head 103 may be a gamma beam, an X-ray beam, a proton beam or other beams.

In some embodiments, the imaging apparatus 200 may generate at least one two-dimensional slice image of the target object P. The at least one two-dimensional slice image includes a desired target volume of the target object P. The two-dimensional slice image may include at least two of a sagittal plane image, a coronal plane image, and a transverse plane image of the target object P.

For example, the sagittal plane is parallel to a YOZ plane in the IEC coordinates, the coronal plane is parallel to an XOY plane in the IEC coordinates, and the transverse plane is parallel to an XOZ plane in the IEC coordinates. In addition, the two-dimensional slice image may also be a two-dimensional oblique slice image. The two-dimensional oblique slice image is not parallel to and not perpendicular to the XOY plane, the YOZ plane and the XOZ plane in the IEC coordinate system. For example, the two-dimensional oblique section image is obliqued relative to the XOY plane, the YOZ plane, and the XOZ plane in the IEC coordinate system.

In some embodiments, the imaging apparatus 200 may be a magnetic resonance imaging (MRI) apparatus, a Compton scatter imaging apparatus, or other apparatus that may directly obtain a two-dimensional slice image of a desired target volume of a target object, rather than processing the obtained three-dimensional image to obtain the two-dimensional slice image.

In some embodiments, the imaging apparatus 200 is disposed on the gantry 101 of the radiotherapy apparatus 100, and may generate the two-dimensional slice image of the target object during the setup phase before radiotherapy or during the radiotherapy phase in the process of radiotherapy.

For example, in a case where the imaging apparatus 200 is the MRI apparatus, as shown in FIG. 1A, the MRI apparatus may include a first magnet 201A and a second magnet 202A, which may generate a magnetic field for imaging. In a case where the target object P on the positioning support device 102 is located in the magnetic field, a two-dimensional slice image of the target object may be generated. Here, the two-dimensional slice image may include a sagittal plane D1 image and a coronal plane D2 image of the target object P.

For example, in a case where the imaging apparatus 200 is the Compton scattering imaging apparatus, as shown in FIG. 1B, the Compton scattering imaging apparatus includes a ray source 201B, a slit beam collimator 202B, a pinhole collimator or slat collimator 203B and a scattering imager 204B.

In this case, the beam emitted by the ray source 201B is transmitted to the target object P through the slit beam collimator 202B, and is scattered by the target object P to form scattered rays. The scattered rays are received by the scattering imager 204B through the pinhole collimator or slat collimator 203B, so as to form a two-dimensional oblique slice image containing the desired target volume T. The oblique section D3 of the two-dimensional oblique slice image is not parallel to and not perpendicular to the XOY plane, YOZ plane and XOZ plane in the IEC coordinate system.

In some embodiments, as shown in FIG. 1C, the imaging apparatus 200 and the radiotherapy apparatus 100 share a positioning support device 102, and the positioning support device 102 may deliver the target object P into the imaging apparatus 200 and the radiotherapy apparatus 100, respectively.

For example, the positioning support device 102 may include a positioning support base 1021 and a positioning support table 1022. The positioning support base 1021 is used to support the positioning support table 1022, and the target object P may be located on the positioning support table 1022.

In some embodiments, the positioning support base 1021 may be rotatable, so that the positioning support base 1021 may deliver the target object on the positioning support table 1022 into the imaging apparatus 200 and the radiotherapy apparatus 100, respectively.

In some embodiments, the positioning support table 1022 may be rotatable, so that the positioning support table 1022 may deliver the target object on the positioning support table 1022 into the imaging apparatus 200 and the radiotherapy apparatus 100, respectively.

In some embodiments, both of the positioning support base 1021 and the positioning support table 1022 may be rotatable, and may deliver the target object on the positioning support table 1022 into the imaging apparatus 200 and the radiotherapy apparatus 100, respectively.

In some embodiments, the target volume monitoring electronic apparatus 300 is communicatively connected to the imaging apparatus 200, and the target volume monitoring electronic apparatus 300 is used to obtain the two-dimensional slice image of the target object generated by the imaging apparatus 200. In this case, the target volume monitoring electronic apparatus 300 may perform the target volume monitoring method provided in some of the following embodiments, and output the registration result.

The target volume monitoring electronic apparatus 300 may be a computer apparatus having a graphical user interface (GUI), which may obtain the three-dimensional reference image and the at least one two-dimensional slice image of the target object from the imaging apparatus 200, and register the at least one two-dimensional slice image with the three-dimensional reference image, so as to obtain the registration result for monitoring the position of the desired target volume.

The radiotherapy control electronic apparatus 400 is communicatively connected to the target volume monitoring electronic apparatus 300, and is used to obtain the registration result and control the operation of the radiotherapy apparatus according to the registration result. For example, the radiotherapy control electronic apparatus 400 may control the movement of the positioning support device 102 of the radiotherapy apparatus 100. Alternatively, the radiotherapy control electronic apparatus 400 may control the radiotherapy head of the radiotherapy apparatus 100 to stop emitting the beam, etc.

In some embodiments of the present disclosure, at least one of the target volume monitoring electronic apparatus 300 or the radiotherapy control electronic apparatus 400 may be a server. For example, at least one of the target volume monitoring electronic apparatus 300 or the radiotherapy control electronic apparatus 400 may be an independent server, may be a server network or server cluster composed of servers.

In some embodiments of the present disclosure, communication between the target volume monitoring electronic apparatus 300 and the imaging apparatus 200, communication between the target volume monitoring electronic apparatus 300 and the radiotherapy control electronic apparatus 400, and communication between the radiotherapy control electronic apparatus 400 and the radiotherapy apparatus 100 may be implemented through any communication methods.

For example, the methods for implementing communication include, but are not limited to, mobile communications based on the 3rd Generation Partnership Project (3GPP), Long Term Evolution (LTE), and Worldwide Interoperability for Microwave Access (WiMAX), or computer network communications based on the TCP/IP Protocol Suite (TCP/IP), User Datagram Protocol (UDP), and Digital Imaging and Communications in Medicine (DICOM).

It will be understood by those skilled in the art that, the application environments shown in FIGS. 1A to 1C are merely application scenarios in some embodiments of the present disclosure, and do not constitute a limitation on the application scenario of the present disclosure. Other application environments may further include more or fewer devices than those shown in FIGS. 1A to 1C. It will be understood that the system may also include one or more other electronic apparatuses that may process data, such as a treatment planning system (TPS) electronic apparatus, an oncology information system (OIS), etc., the present disclosure is not limited thereto.

In addition, the radiotherapy system may further include a storage device. The storage device may be communicatively connected to the imaging apparatus 200 and used to store the at least one two-dimensional slice image of the target object. Accordingly, the target volume monitoring electronic apparatus 300 may obtain at least one two-dimensional slice image of the target object from the storage device.

It will be noted that the scenario diagrams for the radiotherapy system shown in FIGS. 1A to 1C are merely examples. The radiotherapy system and scenarios described in some embodiments of the present disclosure are intended to illustrate the technical solutions of some embodiments of the present disclosure clearly, and do not constitute a limitation on the technical solutions provided by some embodiments of the present disclosure. It will be understood by those skilled in the art that, with the evolution of radiotherapy and the emergence of new business scenarios, the technical solutions provided by some embodiments of the present disclosure are also applicable to similar technical problems.

Hereinafter, the method provided by some embodiments of the present disclosure will be introduced based on the implementation environment shown in FIGS. 1A to 1C.

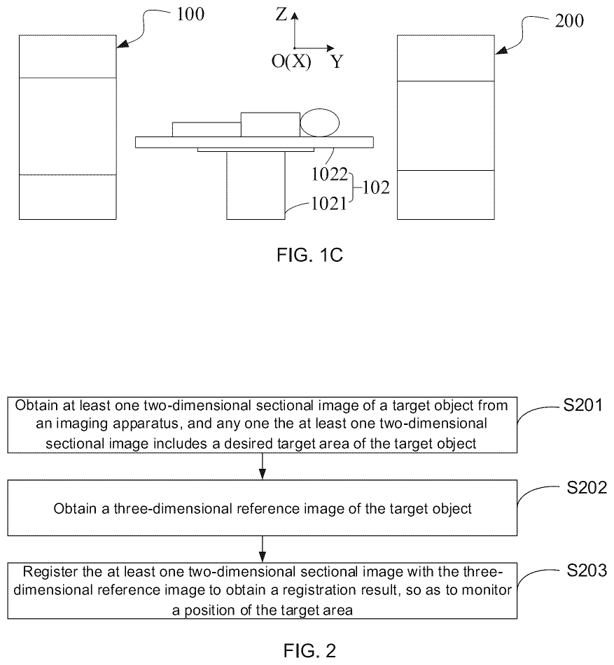

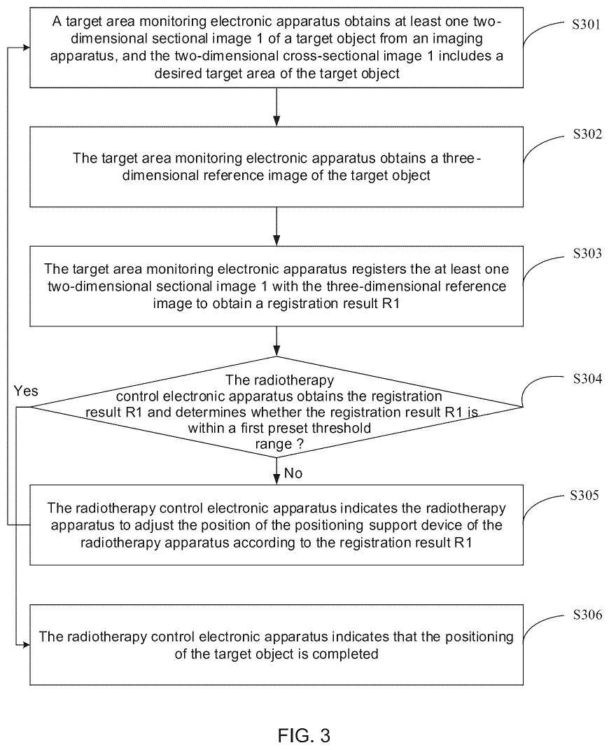

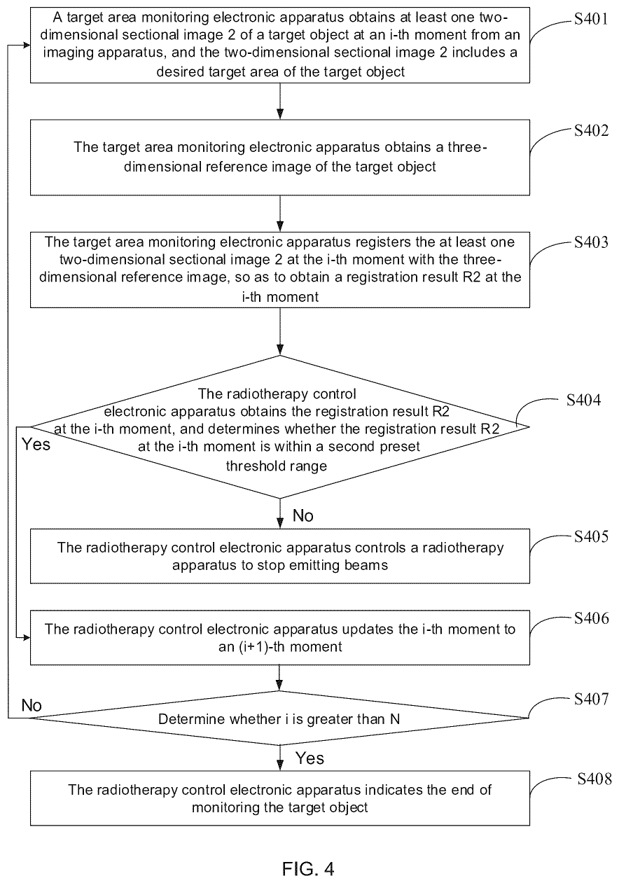

FIG. 2 is a schematic flow chart of a target volume monitoring method, in accordance with some embodiments of the present disclosure. In some embodiments, the target volume monitoring method is performed by an electronic apparatus. For example, the electronic apparatus may be the target volume monitoring electronic apparatus 300 shown in FIG. 1A or FIG. 1B. As shown in FIG. 2, the method includes the following step S201 to step S203.

In S201, at least one two-dimensional slice image of a target object is obtained from an imaging apparatus, and the at least one two-dimensional slice image includes a desired target volume of the target object.

It can be understood that the imaging apparatus may quickly image the target object. In this way, the imaging apparatus may directly generate the at least one two-dimensional slice image of the target object, and the two-dimensional slice image includes the desired target volume of the target object, thus, it can shorten the time of image processing (e.g., the digital reconstruction and other processing) to obtain the at least one two-dimensional slice image.

Accordingly, the target volume monitoring electronic apparatus may quickly obtain at least one two-dimensional slice image of the target object including the desired target volume from the imaging apparatus.

Here, the imaging apparatus may be an MRI apparatus, a Compton scatter imaging apparatus or other apparatus that may directly obtained at least one two-dimensional slice image of the target object including the desired target volume. The target object may be a patient located on the positioning support device or a phantom simulating a patient positioned on the positioning support device, and correspondingly, the desired target volume may be a tumor target volume of the patient or a simulated target volume in the phantom.

Each two-dimensional slice image includes the desired target volume of the target object, that is, the two-dimensional slice image is a two-dimensional image passing through the section of the desired target volume of the target object. In this way, subsequently, the two-dimensional slice image may be registered with the reference three-dimensional image for the desired target volume.

It will be noted that the at least one two-dimensional slice image may include a two-dimensional slice image including the desired target volume of the target object. That is, any two-dimensional image of the target object passes through the section of the desired target volume. The least one two-dimensional slice image may also be two or more two-dimensional images of the target object including different sections of the desired target volume, that is, any two or more slice images of the target object pass through the desired target volume.

In some embodiments, in a case where the at least one two-dimensional slice image includes a single two-dimensional slice image, the single two-dimensional slice image may be a two-dimensional oblique slice image. The two-dimensional oblique slice image is not parallel to and not perpendicular to each coordinate plane in a preset coordinate system.

In this way, by registering the single two-dimensional oblique slice image with the three-dimensional reference image, the offset of the target object in at least three horizontal directions in the preset coordinate system may be determined, thereby achieving the monitoring of the desired target volume.

For example, the preset coordinate system may be the IEC coordinate system, and the two-dimensional oblique slice image may be a two-dimensional slice image that is not parallel to and not perpendicular to an XOY plane, a YOZ plane, and an XOZ plane in the IEC coordinate system. It will be noted that the two-dimensional slice image may include other slice images other than the two-dimensional oblique slice image.

In some embodiments, in a case where the at least one two-dimensional slice image includes a plurality of two-dimensional slice images, the plurality of two-dimensional slice images may include a first two-dimensional slice image and a second two-dimensional slice image. The first two-dimensional slice image and the second two-dimensional slice image are images of two different sections. The first two-dimensional slice image and the second two-dimensional slice image may be parallel or perpendicular to at least one coordinate plane in the preset coordinate system.

In this way, at least two different two-dimensional slice images need to be registered with the three-dimensional reference image to determine the offset of the target object in at least three horizontal directions in the preset coordinate system, thereby implementing the monitoring of the desired target volume.

For example, the preset coordinate system is the IEC coordinate system, the first two-dimensional slice image may be a two-dimensional slice image parallel to or perpendicular to any one of the XOY plane, YOZ plane and XOZ plane in the IEC coordinate system, and the second two-dimensional slice image may be a two-dimensional slice image parallel to any one of the XOY plane, YOZ plane and XOZ plane in the IEC coordinate system.

It will be noted that the plurality of two-dimensional slice images may include other slice images other than the first two-dimensional slice image and the second two-dimensional slice image.

It will be noted that there may be a preset angle between the first two-dimensional slice image and the second two-dimensional slice image. Here, the preset angle may be equal to or less than 90 degrees.

In a case where the preset angle is equal to 90 degrees, the first two-dimensional slice image and the second two-dimensional slice image may be any two of the coronal plane image, the sagittal plane image or the transverse plane image of the target object. For example, the first two-dimensional slice image may be the sagittal image of the target object, and the second two-dimensional slice image may be the coronal image of the target object.

In a case where the preset angle is less than 90 degrees, the first two-dimensional slice image may be the sagittal image, the coronal image or the transverse image of the target object, and the second two-dimensional slice image may be a two-dimensional slice image that forms the preset angle with the first two-dimensional slice image. For example, the first two-dimensional slice image may be the sagittal image or the coronal image of the target object, and the second two-dimensional slice image may be a two-dimensional slice image that forms the preset angle with the first two-dimensional slice image.

In step S202, a three-dimensional reference image of the target object is obtained.

The target volume monitoring electronic apparatus may obtain the three-dimensional reference image of the target object, and the three-dimensional reference image includes the desired target volume of the target object.

Here, the three-dimensional reference image may be set to different types according to different types of imaging apparatuses for obtaining the two-dimensional slice image.

For example, in a case where the imaging apparatus is the MRI apparatus, the at least one two-dimensional slice image is at least one of the sagittal image, the coronal image, the two-dimensional oblique section or other two-dimensional slice images of the target object obtained by the MRI apparatus, and the three-dimensional reference image of the target object may be a three-dimensional MRI image.

For example, in a case where the imaging apparatus is the Compton scattering imaging apparatus, the at least one two-dimensional slice image is at least one of the sagittal image, the coronal image, the two-dimensional oblique section, or other two-dimensional slice images of the target object obtained by the Compton scattering imaging apparatus, and the three-dimensional reference image of the target object may be a three-dimensional CT image or a three-dimensional MRI image.

It will be noted that step S202 may be performed after step S201, and of course, the step S202 may also be performed before the step S201.

In step S203, the at least one two-dimensional slice image is registered with the three-dimensional reference image to obtain a registration result, so as to monitor the position of the desired target volume.

It can be understood that after obtaining the image to be registered (i.e., the at least one two-dimensional slice image and the three-dimensional reference image), the target volume monitoring electronic apparatus may further register the at least one two-dimensional slice image with the three-dimensional reference image to obtain the corresponding registration result, in this way, it is possible to monitor the position of the desired target volume according to the registration result.

Here, the registration refers to the registration between the two-dimensional image and the three-dimensional image. In essence, it is to find a tomographic slice that is most similar to the two-dimensional slice image (e.g., the desired target volume) in the three-dimensional reference image, so as to obtain the offset of the two-dimensional slice image relative to the tomographic slice, that is, the registration result. The registration result is at least a three-dimensional offset, that is, the horizontal offset in the horizontal direction of each coordinate axis in the preset coordinate system. Of course, the registration result may further include a rotational offset in the rotation direction around each coordinate axis of the preset coordinate system.

In the technical solution provided by some embodiments of the present disclosure, the at least one two-dimensional slice image of the desired target volume of the target object is obtained from the imaging apparatus, and the at least one two-dimensional slice image of the target object is registered with the three-dimensional reference image of the target object to obtain the registration result, thereby monitoring the position of the desired target volume based on the registration result.

Due to the fast speed of obtaining the two-dimensional slice images, the monitoring method may be used for positioning before radiotherapy or for real time monitoring during radiotherapy. In addition, the image quality of the two-dimensional slice images is high, thereby improving the monitoring accuracy of the position of the target volume and further improving the accuracy of radiotherapy.

The monitoring method will be explained in detail below by considering the above monitoring method used for one of the positioning before radiotherapy or the real time monitoring during radiotherapy as an example.

In the setup phase before radiotherapy, after the target object is fixed to the positioning support device of the radiotherapy apparatus, the target volume monitoring method further includes step S301 to step S306, so as to move the position of the target object according to the registration result.

In step S301, the target volume monitoring electronic apparatus obtains at least one two-dimensional slice image 1 of the target object from the imaging apparatus, and the two-dimensional slice image 1 includes the desired target volume of the target object.

In step S302, the target volume monitoring electronic apparatus obtains a three-dimensional reference image of the target object.

In step S303, the target volume monitoring electronic apparatus registers the at least one two-dimensional slice image 1 with the three-dimensional reference image to obtain a registration result R1.

In step S304, the radiotherapy control electronic apparatus obtains the registration result R1 and determines whether the registration result R1 is within a first preset threshold range.

Here, the first preset threshold range is a preset threshold range, for example, the first preset threshold range is within minus 1 millimetre to 1 millimetre, inclusive (i.e., [−1 mm, +1 mm]).

In step S305, if it is determined that the registration result R1 is outside the first preset threshold range, the radiotherapy control electronic apparatus indicates the radiotherapy apparatus to adjust the position of the positioning support device of the radiotherapy apparatus according to the registration result 1 (that is, move the position of the target object according to the registration result), and step S301 is re-performed.

In step S306, if it is determined that the registration result R1 is within the first preset threshold range, the radiotherapy control electronic apparatus indicates that the positioning of the target object is completed.

After the target object is positioned, the radiotherapy phase may begin. During the radiotherapy phase, assuming that the radiotherapy phase includes N (N being an integer greater than 0) moments, the target volume monitoring method further includes that the following step S401 to step S408 are performed at an i-th moment (i being an integer greater than or equal to 0 and less than or equal to N) in the N moments, so as to monitor the position of the desired target volume in real time according to the registration result.

In step S401, the target volume monitoring electronic apparatus obtains at least one two-dimensional slice image 2 of the target object at the i-th moment from the imaging apparatus, and the two-dimensional slice image 2 includes the desired target volume of the target object.

In step S402, the target volume monitoring electronic apparatus obtains a three-dimensional reference image of the target object.

In step S403, the target volume monitoring electronic apparatus registers the at least one two-dimensional slice image 2 at the i-th moment with the three-dimensional reference image, so as to obtain a registration result R2 at the i-th moment.

In step S404, the radiotherapy control electronic apparatus obtains the registration result R2 at the i-th moment, and determines whether the registration result R2 at the i-th moment is within a second preset threshold range.

Here, the second preset threshold range is a preset threshold range, which may be the same as the first preset threshold range or may be different from the first preset threshold range. For example, the second preset threshold range is within minus 1millimetre to 1 millimetre, inclusive (i.e., [−1 mm, +1 mm]).

In step S405, if it is determined that the registration result R2 at the i-th moment is outside the preset threshold range, the radiotherapy control electronic apparatus controls the radiotherapy apparatus to stop emitting beams.

In step S406, if it is determined that the registration result R2 at the i-th moment is within the preset threshold range, the radiotherapy control electronic apparatus updates the i-th moment to an (i+1)-th moment.

In step S407, the radiotherapy control electronic apparatus determines whether i is greater than N. If so, step S408 is performed; if not, step S401 is re-performed.

It can be understood that in a case where the radiotherapy control electronic apparatus determines that i is less than or equal to N, it indicates that the radiotherapy phase has not ended and the position of the target object needs to continue to be monitored in real time. In this case, step S401 is re-performed to continue to obtain the registration result R2 at the i-th moment (essentially a registration result R2 at a next moment after the i-th moment).

In step S408, in a case where the radiotherapy control electronic apparatus determines that i is greater than N, it indicates that the radiotherapy phase is ended and there is no need to continue to monitor the position of the target object in real time, so that the radiotherapy control electronic apparatus indicates the end of monitoring the target object.

It will be noted that in actual applications, it is possible to choose to monitor the target object only in the setup phase before radiotherapy, or only in the radiotherapy phase, according to the needs of radiotherapy. Of course, it is also possible to monitor the entire process of radiotherapy for the target object both in the setup phase and in the radiotherapy phase.

In some embodiments, the section of the two-dimensional slice image obtained in the setup phase may be the same as the section of the two-dimensional slice image obtained in the radiotherapy phase. Of course, the section of the two-dimensional slice image obtained in the setup phase may also be different from the section of the two-dimensional slice image obtained in the radiotherapy phase.

For example, the at least one two-dimensional slice image 1 is a first two-dimensional oblique slice image, and the at least one two-dimensional slice image 2 is a second two-dimensional oblique slice image, where the oblique sections of the first two-dimensional oblique slice image and the second two-dimensional oblique slice image are different from each other, and accordingly, the section of the first two-dimensional oblique slice image is different from the section of the second two-dimensional oblique slice image.

For example, the at least one two-dimensional slice image 1 includes a first two-dimensional slice image and a second two-dimensional slice image, and the at least one two-dimensional slice image 2 includes a third two-dimensional slice image and a fourth two-dimensional slice image. In this case, the section corresponding to at least one of the first two-dimensional slice image and the second two-dimensional slice image is different from the section corresponding to at least one of the third two-dimensional slice image and the fourth two-dimensional slice image.

In some embodiments, a spatial resolution of the two-dimensional slice image obtained during the setup phase may be equal to a spatial resolution of the two-dimensional slice image obtained during the radiotherapy phase. Of course, the spatial resolution of the two-dimensional slice image obtained during the setup phase may also be higher than the spatial resolution of the two-dimensional slice image obtained during the radiotherapy phase, thus making the positioning accuracy higher.

For example, considering the imaging apparatus as the MRI apparatus as an example, the steps of the above monitoring method used in the positioning before radiotherapy and radiotherapy will be explained in detail.

In the setup phase before radiotherapy, the target volume monitoring method includes step S501 to step S506.

In step S501, the target volume monitoring electronic apparatus obtains a sagittal image and a coronal image of the target object from the MRI apparatus, and the sagittal image and the coronal image each include the desired target volume of the target object.

In step S502, the target volume monitoring electronic apparatus obtains a three-dimensional reference image of the target object.

In step S503, the target volume monitoring electronic apparatus registers the sagittal plane image and the coronal plane image with the three-dimensional reference image, so as to obtain a registration result.

In step S504, the radiotherapy control electronic apparatus obtains the registration result and determines whether the registration result is within a preset threshold range.

Here, the preset threshold range is [−1 mm, +1 mm].

In step S505, if it is determined that the registration result R1 is outside the first preset threshold range, the radiotherapy control electronic apparatus instructs the radiotherapy apparatus to adjust the position of the positioning support device of the radiotherapy apparatus according to the registration result 1. That is, the position of the target object is moved according to the registration result, and step S501 is re-performed.

In step S506, if it is determined that the registration result R1 is within the first preset threshold range, the radiotherapy control electronic apparatus indicates that the positioning of the target object is completed.

The target object is monitored during the setup phase, and after the positioning is completed, the radiotherapy phase may begin. During the radiotherapy phase, the target volume monitoring method further includes step S507 to step S514.

In step S507, the target volume monitoring electronic apparatus obtains the two-dimensional oblique slice image of the target object at the i-th moment in the radiotherapy phase from the MRI apparatus; the two-dimensional oblique slice image includes the desired target volume of the target object, and the spatial resolution of the two-dimensional oblique slice image is lower than or equal to any one of the spatial resolution of the sagittal image and the spatial resolution of the coronal image.

In step S508, the target volume monitoring electronic apparatus obtains the three-dimensional reference image of the target object.

In step S509, the target volume monitoring electronic apparatus registers the two-dimensional oblique slice image at the i-th moment with the three-dimensional reference image to obtain the registration result at the i-th moment.

In step S510, the radiotherapy control electronic apparatus obtains the registration result at the i-th moment and determines whether the registration result at the i-th moment is within the preset threshold range.

In step S511, if it is determined that the registration result at the i-th moment is outside the preset threshold range, the radiotherapy control electronic apparatus controls the radiotherapy apparatus to stop emitting beams, and the monitoring is stopped.

In step S512, if it is determined that the registration result at the i-th moment is within a preset threshold range, the radiotherapy control electronic apparatus updates the i-th moment to the (i+1)-th moment.

In step S513, the radiotherapy control electronic apparatus determines whether i is greater than N. In a case where the radiotherapy control electronic apparatus determines that i is less than or equal to N, step S507 is re-performed.

In step S514, in a case where the radiotherapy control electronic apparatus determines that i is greater than N, there is no need to continue to monitor the position of the target object in real time, and the monitoring of the target object is ended.

Some embodiments of the present disclosure further provide a target volume monitoring electronic apparatus, which includes at least one processor, and a memory communicatively connected to the at least one processor. For example, the memory stores instructions executable by the at least one processor, and the instructions are executed by the at least one processor, so as to enable the at least one processor to execute the target volume monitoring method provided by some embodiments of the present disclosure.

Some embodiments of the present disclosure further provide a non-transitory computer-readable storage medium storing computer instructions. For example, the computer instructions are used to enable the electronic apparatus to execute the target volume monitoring method provided by some embodiments of the present disclosure.

Some embodiments of the present disclosure further provide a computer program product, which includes a computer program that, when executed by a processor, implements the target volume monitoring method provided by some embodiments of the present disclosure.

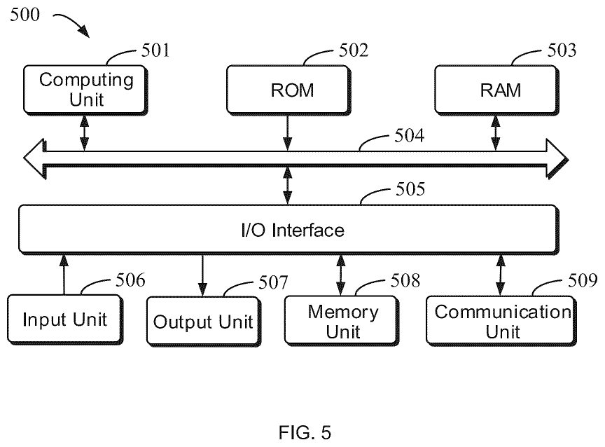

In some embodiments, the target volume monitoring electronic apparatus may be the above-mentioned server shown in FIG. 1A and FIG. 1B. FIG. 5 is a block diagram of a target volume monitoring electronic apparatus 500 used to implement the target volume monitoring method, in accordance with some embodiments of the present disclosure.

The target volume monitoring electronic apparatus 500 is intended to represent various forms of digital computers, such as a laptop computer, a desktop computer, a workstation, a personal digital assistant, a server, a blade server, a mainframe computer, and other suitable computers.

The target volume monitoring electronic apparatus 500 may also represent various forms of mobile devices, such as a personal digital assistant, a cellular phone, a smart phone, a wearable device, and other similar computing devices.

The components shown herein, their connections and relationships, and their functions are examples only and are not intended to limit implementations of some embodiments of the present disclosure.

As shown in FIG. 5, the target volume monitoring electronic apparatus 500 includes a computing unit 501. The computing unit 501 may execute various appropriate actions and processes according to a computer program stored in a read only memory (ROM) 502 or a computer program loaded from a memory unit 508 to a random access memory (RAM) 503.

Various programs and data required for the operation of the target volume monitoring electronic apparatus 500 may further be stored in the RAM 503. The calculation unit 501, the ROM 502, and the RAM 503 are connected to each other through a bus 504. An input/output (I/O) interface 505 is further connected to the bus 504.

A plurality of components in the target volume monitoring electronic apparatus 500 are connected to the I/O interface 505. The plurality of components include an input unit 506, such as a keyboard and a mouse; an output unit 507, such as various types of displays and speakers; a memory unit 508, such as a disk and an optical disk; and a communication unit 509, such as a network card, a modem, and a wireless communication transceiver.

The communication unit 509 allows the target volume monitoring electronic apparatus 500 to exchange information or data with other devices through a computer network such as the Internet and at least one of various telecommunication networks.

The computing unit 501 may be at least one of various general purpose processing components or special purpose processing components having processing and computing capabilities.

Some examples of computing unit 501 include, but are not limited to, a central processing unit (CPU), a graphics processing unit (GPU), various dedicated artificial intelligence (Al) computing chips, various computing units that run machine learning model algorithms, a digital signal processor (DSP), and any appropriate processor, controller, microcontroller, etc. The computing unit 501 executes the various methods and processes described above, such as the target volume monitoring method.

For example, in some embodiments, the target volume monitoring method may be implemented as a computer software program tangibly included in a machine-readable medium, such as in the memory unit 508.

In some embodiments, part or all of the computer program may be loaded onto the target volume monitoring electronic apparatus 500 through at least one of the ROM 502 or the communication unit 509.

In some embodiments, part or all of the computer program may be installed onto the target volume monitoring electronic apparatus 500 through at least one of the ROM 502 or the communication unit 509.

In some embodiments, part or all of the computer program may be loaded and installed onto the target volume monitoring electronic apparatus 500 through at least one of the ROM 502 or the communication unit 509.

In a case where the computer program is loaded into the RAM 503 and executed by the computing unit 501, one or more steps of the target volume monitoring method described above may be executed. Alternatively, in some other embodiments, the computing unit 501 may be configured to perform the target volume monitoring method in any other appropriate method (e.g., by means of firmware).

Various implementations of the systems and techniques described above in the context may be in one of a digital electronic circuit system, an integrated circuit system, a field programmable gate array (FPGA), an application specific integrated circuit (ASIC), an application specific standard parts (ASSP), a system on a chip (SOC), a complex programmable logic device (CPLD), a computer hardware, a firmware, a software, or a combination thereof.

In some embodiments, these various embodiments may include implementations in one or more computer programs.

In some embodiments, the one or more computer programs are executable on a programmable system including at least one programmable processor. In some embodiments, the one or more computer programs may be interpreted on a programmable system including at least one programmable processor. In some embodiments, the one or more computer programs are executable and interpreted on a programmable system including at least one programmable processor.

The programmable processor, which may be a special purpose or general purpose programmable processor, may receive data and instructions from a memory system, at least one input device, and at least one output device, and transmit data and instructions to the memory system, the at least one input device, and the at least one output device.

Program codes for implementing the methods in some embodiments of the present disclosure may be written in any combination of one or more programming languages. Such program codes may be provided to a processor or controller of a general purpose computer, a special purpose computer, or other programmable data processing device such that the program code, when executed by the processor or controller, causes the functions or operations set forth in at least one of the flowcharts or the block diagrams to be performed.

The program codes may be executed entirely on the machine, partially on the machine, partially on the machine as a stand-alone software package and partially on a remote machine, or entirely on a remote machine or server.

In some embodiments of the present disclosure, a machine-readable medium may be a tangible medium that may contain or store a program for use by or in connection with instructions to execute system, device, or apparatus.

The machine-readable medium may be a machine-readable signal medium or a machine-readable storage medium. Machine-readable media may include, but are not limited to, electronic, magnetic, optical, electromagnetic, infrared, or semiconductor systems, apparatuses or devices, or any suitable combination of the foregoing.

For example, the machine-readable storage medium may include an electrical connection based on one or more wires, a portable computer disk, a hard disk, a random-access memory, a read-only memory, an erasable programmable read only memory (EPROM or flash memory), an optical fiber, a convenient compact disc read-only memory (CD-ROM), an optical storage device, a magnetic storage device, or any suitable combination of the foregoing.

To provide interaction with a user, the systems and techniques described herein may be implemented on a computer. The computer has: a display device, such as a cathode ray tube (CRT) or liquid crystal display (LCD) monitor, for displaying information to the user; and a keyboard and a pointing device (e.g., a mouse or trackball) through which the user may provide input to the computer.

Other kinds of devices may also be used to provide interaction with the user. For example, the feedback provided to the user may be any form of sensory feedback (e.g., visual feedback, auditory feedback, or tactile feedback); and may be provided in any form, including acoustic input, speech input, or tactile input) to receive input from the user.

The systems and techniques described herein may be implemented in a computing system including back-end components (e.g., as a data server), or a computing system including middleware components (e.g., an application server), or a computing system including front-end components (e.g., a user's computer having a graphical user interface or web browser through which the user may interact with implementations of the systems and technologies described herein), or a computing system including any combination of such backend components, middleware components, or front-end components.

The components of the system may be interconnected by any form or medium of digital data communication (e.g., a communications network). Examples of communication networks include: local area network (LAN), wide area network (WAN), and the Internet.

Computer systems may include clients and servers. Clients and servers are generally remote from each other and typically interact over a communications network. The relationship of client and server is created by computer programs running on corresponding computers and having a client-server relationship with each other. The server may be a cloud server, or a distributed system server, or a server combined with a blockchain.

It should be understood that various forms of the process shown above may be used, with steps reordered, added or deleted. For example, various steps described in the present disclosure may be executed in parallel, sequentially, or in a different order. As long as the desired results of the technical solution of the present disclosure can be achieved, the present disclosure is no limited thereto.

The above descriptions are merely specific implementation manners of the present disclosure, but the protection scope of the present disclosure is not limited thereto. It will be understood by those skilled in the art that various modifications, combinations, sub-combinations and substitutions may be made depending on design requirements and other factors. Any modifications, equivalent substitutions, improvements, etc. made within the spirit and principles of the present disclosure shall be included in the protection scope of the present disclosure.

What is claimed is:

1. A target volume monitoring method, comprising:
obtaining at least one two-dimensional slice image of a target object from an imaging apparatus; wherein any one of the at least one two-dimensional slice image includes a desired target volume of the target object;
obtaining a three-dimensional reference image of the target object; and
registering the at least one two-dimensional slice image with the three-dimensional reference image to obtain a registration result, so as to monitor a position of the desired target volume.

2. The method according to claim 1, wherein the at least one two-dimensional slice image includes a two-dimensional oblique slice image, and the two-dimensional oblique slice image is not parallel to or perpendicular to each coordinate plane in a preset coordinate system.

3. The method according to claim 1, wherein the at least one two-dimensional slice image includes a first two-dimensional slice image and a second two-dimensional slice image, and the first two-dimensional slice image and the second two-dimensional slice image each are parallel or perpendicular to at least one coordinate plane in a preset coordinate system.

4. The method according to claim 3, wherein there is a preset angle between the first two-dimensional slice image and the second two-dimensional slice image, and the preset angle is equal to or less than 90 degrees.

5. The method according to claim 4, wherein the first two-dimensional slice image and the second two-dimensional slice image satisfy one of the following:
the preset angle is equal to 90 degrees, the first two-dimensional slice image is a sagittal image of the target object, and the second two-dimensional slice image is a coronal image of the target object; and
the preset angle is less than 90 degrees, the first two-dimensional slice image is one of the sagittal image and the coronal image of the target object, and the second two-dimensional slice image is a two-dimensional slice image that has the preset angle with the first two-dimensional slice image.

6. The method according to claim 1, further satisfying at least one of the following:
setup phase performing the monitoring method, during the setup phase, so as to move a position of the target object according to the registration result; or
radiotherapy phase performing the monitoring method, during the radiotherapy phase, so as to monitor the position of the desired target volume in real time according to the registration result.

7. The method according to claim 6, wherein a section of the two-dimensional slice image obtained in the setup phase is different from a section of the two-dimensional slice image obtained in the radiotherapy phase.

8. The method according to claim 6, wherein a spatial resolution of the two-dimensional slice image obtained in the setup phase is higher than a spatial resolution of the two-dimensional slice image obtained in the radiotherapy phase.

9. A target volume monitoring electronic apparatus, comprising:
at least one processor; and
a memory communicatively connected to the at least one processor; wherein
the memory stores instructions executable by the at least one processor, and the instructions is executed by the at least one processor, so that the at least one processor is capable of executing:
obtaining at least one two-dimensional slice image of a target object from an imaging apparatus; wherein any one of the at least one two-dimensional slice image includes a desired target volume of the target object;
obtaining a three-dimensional reference image of the target object; and
registering the at least one two-dimensional slice image with the three-dimensional reference image to obtain a registration result, so as to monitor a position of the desired target volume.

10. The target volume monitoring electronic apparatus according to claim 9, wherein the at least one two-dimensional slice image includes a two-dimensional oblique slice image, and the two-dimensional oblique slice image is not parallel to or perpendicular to each coordinate plane in a preset coordinate system.

11. The target volume monitoring electronic apparatus according to claim 9, wherein the at least one two-dimensional slice image includes a first two-dimensional slice image and a second two-dimensional slice image, and the first two-dimensional slice image and the second two-dimensional slice image each are parallel or perpendicular to at least one coordinate plane in a preset coordinate system.

12. The target volume monitoring electronic apparatus according to claim 11, wherein there is a preset angle between the first two-dimensional slice image and the second two-dimensional slice image, and the preset angle is equal to or less than 90 degrees.

13. The target volume monitoring electronic apparatus according to claim 12, wherein the first two-dimensional slice image and the second two-dimensional slice image satisfy one of the following:
the preset angle is equal to 90 degrees, the first two-dimensional slice image is a sagittal image of the target object, and the second two-dimensional slice image is a coronal image of the target object; and
the preset angle being less than 90 degrees, the first two-dimensional slice image being one of the sagittal image and the coronal image of the target object, and the second two-dimensional slice image is a two-dimensional slice image that has the preset angle with the first two-dimensional slice image.

14. The target volume monitoring electronic apparatus according to claim 9, satisfying at least one of the following:
the target volume monitoring electronic apparatus being configured to: execute a monitoring method during a setup phase, so as to move the position of the target object according to the registration result; or
the target volume monitoring electronic apparatus being configured to: execute the monitoring method during the radiotherapy phase, so as to monitor the position of the desired target volume in real time according to the registration result.

15. The target volume monitoring electronic apparatus according to claim 14, wherein a section of the two-dimensional slice image obtained in the setup phase is different from a section of the two-dimensional slice image obtained in the radiotherapy phase.

16. The target volume monitoring electronic apparatus according to claim 14, wherein a spatial resolution of the two-dimensional slice image obtained in the setup phase is higher than a spatial resolution of the two-dimensional slice image obtained in the radiotherapy phase.

17. A radiotherapy system, comprising:
a radiotherapy apparatus;
an imaging apparatus configured to generate at least one two-dimensional slice image of a target object;
a target volume monitoring electronic apparatus configured to execute the target volume monitoring method according to claim 1 and output a registration result; and
a radiotherapy control electronic apparatus configured to obtain the registration result and control operation of the radiotherapy apparatus according to the registration result.

18. The system according to claim 17, further comprising a positioning support device, wherein the imaging apparatus and the radiotherapy apparatus share the positioning support device;
wherein the imaging apparatus satisfies one of the following:
the imaging apparatus is disposed on a gantry of the radiotherapy apparatus; or
the positioning support device is capable of delivering the target object into the imaging apparatus and the radiotherapy apparatus.

19. A non-transitory computer-readable storage medium storing computer program instructions that, when executed by an electronic apparatus, cause the electronic apparatus to implement the target volume monitoring method according to claim 1.

20. A computer program product including computer program that, when executed by a processor, cause the processor to implement the target volume monitoring method according to claim 1.
